(19) United States
(12) Patent Application Publication
(10) Pub. No.: US 2016/0317416 A1
(43) Pub. Date: Nov. 3, 2016

(54) COSMETIC COMPOSITION COMPRISING AN OIL, A NONIONIC SURFACTANT AND A C-GLYCOSIDE COMPOUND

(71) Applicant: L'OREAL, Paris (FR)

(72) Inventors: Celine Derrips, Chilly Mazarin (FR); Chantal Jouy, Paris (FR); Anne-Laure Bernard, Clark, NJ (US)

(73) Assignee: L'OREAL, Paris (FR)

(21) Appl. No.: 15/107,642

(22) PCT Filed: Dec. 17, 2014
(86) PCT No.: PCT/EP2014/078160

§ 371 Date: Jun. 23, 2016

(30) Foreign Application Priority Data
Dec. 24, 2013 (FR) 1363528

(51) Int. Cl. A61K 8/60; A61K 8/37; A61K 8/34; A61K 8/92; A61Q 19/10; A61Q 1/14; A61Q 3/00; A61Q 5/00; A61Q 19/00; A61K 8/06; A61Q 1/10

(52) U.S. Cl. CPC A61K 8/602; A61K 8/068; A61K 8/37; A61K 8/342; A61K 8/375; A61K 8/92; A61K 8/345; A61K 8/062; A61Q 1/10; A61Q 1/14; A61Q 3/00; A61Q 5/00; A61Q 19/00; A61Q 19/10; A61K 2800/596; A61K 2800/21

(57) ABSTRACT

The present invention relates to a cosmetic composition in the form of a nanoemulsion or microemulsion, comprising: (a) at least one oil; (b) at least one nonionic surfactant with an HLB value from 8.0 to 14.0, preferably from 9.0 to 13.5 and more preferably from 10.0 to 13.0; (c) at least one C-glycoside compound; and (d) water. The cosmetic composition may be in the form of a nanoemulsion or microemulsion with a transparent or slightly translucent appearance.

The present invention relates to a composition, especially a cosmetic composition, in the form of a nanoemulsion or a microemulsion.

Oil-in-water (O/W) emulsions or water-in-oil (W/O) emulsions are known in the field of cosmetics and dermatology, in particular for preparing cosmetic products, such as milks, creams, toners, serums or eaux de toilette. In particular, a fine emulsion such as an O/W nanoemulsion or microemulsion is particularly advantageous in cosmetic products on account of its transparent or slightly translucent appearance.

It is known practice in the field of cosmetics or dermatology to use oil-in-water (O/W) emulsions. These emulsions, which consist of an oily phase (or a lipophilic phase) dispersed in an aqueous phase, have an outer aqueous phase and are therefore products that are more pleasant to use on account of the fresh sensation they afford. However, they have the drawback of lacking stability when the amount of oil present is too high. At the present time, for certain applications, it is advantageous to have a large amount of oils, given that oils give the skin a comfortable feel, nourish it and may also remove makeup therefrom when these oils have makeup-removing properties.

Furthermore, it is advantageous to have fine emulsions, i.e. emulsions in which the oily phase is in the form of very small droplets, i.e. droplets less than 4 μm in size, given that these fine emulsions afford a pleasant cosmetic sensation and are generally more stable than coarse emulsions.

These emulsions may be prepared, in particular, via the phase inversion temperature technique (PIT emulsions), in which the mean size of the globules constituting the oily phase is within given limits, namely between 0.1 and 4 μm (100 to 4000 nm). The principle of phase inversion temperature (PIT) emulsification is, in theoretical terms, known to those skilled in the art; it was described in 1968 by K. Shinoda (J. Chem. Soc. Jpn., 1968, 89, 435). It has been shown that this emulsification technique allows stable, fine emulsions to be obtained (K. Shinoda and H. Saito, J. Colloid Interface Sci., 1969, 30, 258). This technique was applied in cosmetics as early as 1972 by Mitsui et al. (“Application of the phase-inversion-temperature method to the emulsification of cosmetics”; T. Mitsui, Y. Machida and F. Harusawa, American Cosmet. Perfum., 1972, 87, 33).

The principle of this technique is as follows: an O/W emulsion (introduction of the aqueous phase into the oily phase) is prepared at a temperature which should be above the phase inversion temperature of the system, which is the temperature at which the equilibrium between the hydrophilic and lipophilic properties of the emulsifier(s) used is reached; at a higher temperature, i.e. above the phase inversion temperature (>PIT), the emulsion is of water-in-oil type, and, when it cools down, this emulsion inverts at the phase inversion temperature, to become an emulsion of oil-in-water type, passing beforehand through a microemulsion state. This process makes it readily possible to obtain emulsions with a diameter generally less than 4 μm. The emulsifying surfactants of oil-in-water type conventionally used have an HLB (hydrophilic-lipophilic balance) in the range from 8 to 18. On account of their amphiphilic structure, these emulsifiers are located at the oily phase/aqueous phase interface, and thus stabilize the dispersed oil droplets.

However, it is difficult to produce fine O/W emulsions containing a large amount of oily phase, given that such emulsions have a tendency to become destabilized, this destabilization leading to coalescence and separation of the aqueous and oily phases with release of oil. In order to improve the stability of these emulsions, the concentration of emulsifiers may be increased; however, a high concentration of emulsifiers may lead to a rough, adhesive or tacky feel and to safety concerns with respect to the skin, the eyes and the scalp.

In particular, a fine emulsion such as an O/W nanoemulsion or microemulsion is particularly advantageous in cosmetic products on account of its transparent or slightly translucent appearance.

For example, JP-A-H09-110635 describes a fine emulsion which is formed by using a combination of polyglyceryl fatty acid ester as surfactant and a C₁₀-C₂₂ fatty 2-hydroxy acid. Furthermore, JP-A-H11-71256 describes a fine emulsion which is formed by using a combination of polyglyceryl fatty acid ester and betaine.

However, when certain types of nonionic surfactant are used for preparing fine emulsions, the transparent or slightly translucent appearance of the emulsion and the stability of the emulsion are insufficient.

One object of the present invention is to propose a cosmetic composition in the form of a nanoemulsion or microemulsion that has a transparent or slightly translucent, preferably transparent, appearance.

After detailed investigations, the inventors have discovered that it is possible to produce a stable cosmetic composition in the form of a nanoemulsion or microemulsion that has a transparent or slightly translucent, preferably transparent, appearance of the emulsion, even when a nonionic surfactant was used that made it difficult to form a fine emulsion such as a nanoemulsion or microemulsion.

Consequently, the present invention relates to a cosmetic composition in the form of a nanoemulsion or microemulsion, comprising:
(a) at least one oil;
(b) at least one nonionic surfactant with an HLB value from 8.0 to 14.0, preferably from 9.0 to 13.5 and more preferably from 10.0 to 13.0;
(c) at least one C-glycoside compound preferably represented by formula (I)
(d) water.

Given that the cosmetic composition according to the present invention may have a transparent or slightly translucent appearance, the composition may preferably be used for lotions and the like. Furthermore, the cosmetic composition according to the present invention may produce a pleasant texture and afford moisturizing properties and also increased suppleness. Furthermore, if the dispersed phase is an oily phase and comprises one or more lipophilic or even amphiphilic active components, the oily dispersed phase may function as a vehicle for the active substance and accelerate the penetration of the active components into the skin, or may distribute the active components on the skin.

Furthermore, the present invention also relates to a non-therapeutic process for treating the skin, the hair, mucous membranes, the nails, the eyelashes, the eyelids and/or the scalp, characterized in that the cosmetic composition according to the present invention is applied to the skin, the hair, mucous membranes, the nails, the eyelashes, the eyelids or the scalp.

Furthermore, the present invention also relates to a use of the cosmetic composition according to the present invention as or in care products and/or washing products and/or makeup products and/or makeup-removing products for bodily and/or facial skin and/or mucous membranes and/or the scalp and/or the hair and/or the nails and/or the eyelashes and/or the eyelids.

The cosmetic composition according to the present invention is described in greater detail hereinbelow.

The cosmetic composition according to the present invention comprises at least one oil. According to the present invention, the term “oil” denotes a fatty compound or substance that is in the form of a liquid at room temperature (25° C.) and at atmospheric pressure (760 mmHg). As oils, those generally used in cosmetics may be used alone or in combinations thereof. These oils may be volatile or non-volatile, preferably non-volatile.

The oil may be a non-polar oil such as a hydrocarbon oil, a silicone oil or the like; a polar oil such as a plant or animal oil and an ester oil or an ether oil; or a mixture thereof.

It is preferable for the oil (a) to be chosen from the group consisting of oils of plant origin, animal oils, synthetic oils, silicone oils and hydrocarbon oils.

As examples of plant oils, mention may be made, for example, of linseed oil, camellia oil, macadamia oil, corn oil, castor oil, olive oil, avocado oil, sasanqua oil, safflower oil, jojoba oil, sunflower oil, almond oil, rapeseed oil, sesame oil, soybean oil, groundnut oil, argan oil and apricot kernel oil, and mixtures thereof.

As examples of animal oils, mention may be made, for example, of squalene and squalane.

As examples of synthetic oils, mention may be made of alkanes such as isododecane and isohexadecane, fatty esters, fatty ethers and artificial C6-C22 acid triglycerides.

The fatty esters are preferably liquid esters of linear or branched, saturated or unsaturated C₁-C₂₆ aliphatic monoacids or polyacids and of linear or branched, saturated or unsaturated C₁-C₂₆ aliphatic monoalcohols or polyalcohols, the total number of carbon atoms in the fatty esters being greater than or equal to 10.

Preferably, for the monoalcohol esters, at least one from among the alcohol and the acid is branched.

Among the monoesters of monoacids and of monoalcohols, mention may be made of ethyl palm itate, ethylhexyl palm itate, isopropyl palm itate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols and esters of monocarboxylic, dicarboxylic or tricarboxylic acids and of non-saccharide C₄-C₂₆ dihydroxy, trihydroxy, tetrahydroxy or pentahydroxy alcohols may also be used.

Mention may in particular be made of: diethyl sebacate; isopropyllauryl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

Fatty esters that may be used include sugar esters and diesters of C₆-C₃₀ and preferably C₁₂-C₂₂ fatty acids. It is recalled that the term “sugar” means hydrocarbon-based compounds comprising oxygen containing several alcohol functions, with or without aldehyde or ketone functions, and which comprise at least 4 carbon atoms. These sugars may be monosaccharides, oligosaccharides or polysaccharides.

Examples of suitable sugars that may be mentioned include saccharose (or sucrose), glucose, galactose, ribose, fucose, maltose, fructose, mannose, arabinose, xylose and lactose, and derivatives thereof, in particular alkyl derivatives such as methyl derivatives, for example methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may contain from one to three conjugated or unconjugated double bonds.

The esters according to this variant may also be chosen from monoesters, diesters, triesters, tetraesters and polyesters, and mixtures thereof.

These esters may be, for example, oleates, laurates, palm itates, myristates, behenates, cocoates, stearates, linoleates, linolenates, caprates and arachidonates, or mixtures thereof such as, in particular, oleopalmitate, oleostearate and palm itostearate mixed esters, and also pentaerythrityl tetraethylhexanoate.

More particularly, use is made of monoesters and diesters and in particular sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As preferred examples of fatty esters, mention may be made, for example, of diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palm itate, ethyl palm itate, isopropyl palm itate, ethylhexyl palm itate, isohexyl laurate, hexyl laurate, isocetyl stearate, isopropyl isostearate, isopropyl myristate, isodecyl oleate, glyceryl tris(2-ethylhexanoate), pentaerythrityl tetrakis(2-ethylhexanoate), 2-ethylhexyl succinate and diethyl sebacate, and mixtures thereof.

As examples of artificial triglycerides, mention may be made, for example, of glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made, for example, of linear organopolysiloxanes such as dimethylpolysiloxane, methylphenylpolysiloxane, methylhydrogenopolysiloxane and the like; cyclic organopolysiloxanes such as octamethylcyclotetrasiloxane, decamethylcyclopentasiloxane, dodecamethylcyclohexasiloxane and the like; and mixtures thereof.

Preferably, the silicone oil is chosen from liquid polydialkylsiloxanes, in particular liquid polydimethylsiloxanes (PDMS) and liquid polyorganosiloxanes comprising at least one aryl group.

These silicone oils may also be organomodified. The organomodified silicones that may be used according to the present invention are silicone oils as defined above comprising in their structure one or more organofunctional groups linked via a hydrocarbon-based group.

Organopolysiloxanes are defined in greater detail in Walter Noll's Chemistry and Technology of Silicones (1968), Academic Press. They may be volatile or non-volatile.

Volatile or non-volatile silicone oils, such as volatile or non-volatile polydimethylsiloxanes (PDMS) containing a linear or cyclic silicone chain, which are liquid or pasty at room temperature, in particular cyclopolydimethylsiloxanes (cyclomethicones) such as cyclohexasiloxane; polydimethylsiloxanes containing alkyl, alkoxy or phenyl groups that are pendent or at the end of the silicone chain, the said groups containing from 2 to 24 carbon atoms; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenyl ethyl trimethylsiloxysilicates and polymethylphenylsiloxanes, may be used.

The hydrocarbon-based oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, a liquid paraffin gel, polydecenes and hydrogenated polyisobutenes such as Parleam®, and squalane.

As preferred examples of hydrocarbon-based oils, mention may be made, for example, of linear or branched hydrocarbons such as mineral oil (for example liquid paraffin), paraffin, petroleum jelly or petrolatum, naphthalens and the like; hydrogenated polyisobutene, isoeicosane, and a decene/butene copolymer; and mixtures thereof.

It is also preferable for the oil (a) to be chosen from oils with a molecular weight of less than 600 g/mol.

Preferably, the oil (a) is chosen from fatty esters containing one or more C₁-C₁₂ hydrocarbon-based chains (for example isopropyl myristate, isopropyl palm itate, isononyl isononanoate and ethylhexyl palm itate), hydrocarbon oils (for example isododecane, isohexadecane and squalane), oils of branched and/or unsaturated C₁₂-C₃₀ fatty alcohol type such as octyldodecanol or oleyl alcohol, and fatty ethers such as dicaprylyl ether.

The amount of the oil (a) in the cosmetic composition according to the present invention may be in the range from 0.1% to 50% by weight, preferably from 1% to 40% by weight and more preferably from 5% to 30% by weight relative to the total weight of the composition.

[Nonionic Surfactant]

The cosmetic composition according to the present invention may comprise at least one specific nonionic surfactant.

The nonionic surfactant has an HLB (hydrophilic-lipophilic balance) value of from 8.0 to 14.0, preferably from 9.0 to 13.5 and more preferably from 10.0 to 13.0. If two or more nonionic surfactants are used, the HLB value is determined by the mean weight of the HLB values of all the nonionic surfactants.

The term “HLB” is well known to those skilled in the art, and denotes the hydrophilic-lipophilic balance of a surfactant.

The HLB or hydrophilic-lipophilic balance of the surfactant(s) used according to the invention is the HLB according to Griffin, defined in the publication J. Soc. Cosm. Chem. 1954 (Vol. 5), pages 249-256 or the HLB determined experimentally and as described in the publication from the authors F. Puisieux and M. Seiller, entitled Galenica 5: Les systèmes dispersés—Tome I—Agents de surface et émulsions [Galenica 5: Dispersed systems—Volume I—Surface agents and emulsions]—Chapter IV—Notions de HLB et de HLB critique [Notions of HLB and of critical HLB], pages 153-194—paragraph 1.1.2. Détermination de HLB par voie expérimentale [Experimental determination of HLB], pages 164-180.

It is preferably the calculated HLB values that should be taken into account.

The calculated HLB is defined as being the following coefficient:

calculated HLB=20×molar mass of the hydrophilic part/total molar mass.

For an oxyethylenated fatty alcohol, the hydrophilic part corresponds to the oxyethylene units fused to the fatty alcohol and the calculated HLB value then corresponds to the HLB value according to Griffin (Griffin W. C., J. Soc. Cosmet. Chemists, 5, 249, 1954).

The nonionic surfactant with an HLB value of from 8.0 to 14.0, preferably from 9.0 to 13.5 and more preferably from 10.0 to 13.0 may be chosen from:
(1) surfactants that are fluid at a temperature of less than or equal to 45° C., chosen from esters of at least one polyol chosen from the group formed by a polyethylene glycol comprising from 1 to 60 ethylene oxide units, sorbitan, glycerol comprising from 2 to 30 ethylene oxide units, and polyglycerols comprising from 2 to 12 glycerol units, and of at least one fatty acid comprising at least one saturated or unsaturated, linear or branched C₈-C₂₂ alkyl chain,
(2) mixed esters of fatty acid or of fatty alcohol, of carboxylic acid and of glycerol,
(3) fatty acid esters of sugars and fatty alkyl ethers of sugars,
(4) surfactants that are solid at a temperature of less than or equal to 45° C., chosen from fatty acid esters of glycerol, fatty acid esters of sorbitan and oxyethylenated fatty acid esters of sorbitan, ethoxylated fatty acid ethers and ethoxylated fatty acid esters,
(5) block copolymers of ethylene oxide (A) and of propylene oxide (B), and
(6) silicone surfactants.

The surfactants (1) that are fluid at a temperature of less than or equal to 45° C. may be, in particular:
polyethylene glycol isostearate of molecular weight 400, sold under the name PEG 400 by the company Uniqema;
diglyceryl isostearate, sold by the company Solvay;
glyceryl laurate comprising 2 glycerol units, sold by the company Solvay;
sorbitan oleate, sold under the name Span 80 by the company ICI;
sorbitan isostearate, sold under the name Nikkol SI 10R by the company Nikko; and
α-butylglucoside cocoate or α-butylglucoside caprate, sold by the company Ulice.

The mixed esters of fatty acid or of fatty alcohol (2), of carboxylic acid and of glycerol, which may be used as nonionic surfactant above, may be chosen in particular from the group comprising mixed esters of fatty acid or of fatty alcohol with an alkyl chain containing from 8 to 22 carbon atoms, and of a-hydroxy acid and/or of succinic acid, with glycerol. The α-hydroxy acid may be, for example, citric acid, lactic acid, glycolic acid or malic acid, and mixtures thereof.

The alkyl chain of the fatty acids or fatty alcohols from which are derived the mixed esters that may be used in the emulsion of the invention may be linear or branched, and saturated or unsaturated. They may in particular be stearate, isostearate, linoleate, oleate, behenate, arachidonate, palm itate, myristate, laurate, caprate, isostearyl, stearyl, linoleyl, oleyl, behenyl, myristyl, lauryl or capryl chains, and mixtures thereof.

As examples of mixed esters that may be used in the emulsion of the invention, mention may be made of the mixed ester of glycerol and the mixture of citric acid, lactic acid, linoleic acid and oleic acid (CTFA name: glyceryl citrate/lactate/linoleate/oleate) sold by the company Hüls under the name Imwitor 375; the mixed ester of succinic acid and of isostearyl alcohol with glycerol (CTFA name: isostearyl-diglyceryl succinate) sold by the company Hüls under the name Imwitor 780 K; the mixed ester of citric acid and of stearic acid with glycerol (CTFA name: glyceryl stearate-citrate) sold by the company Hüls under the name Imwitor 370; the mixed ester of lactic acid and of stearic acid with glycerol (CTFA name: glyceryl stearate-lactate) sold by the company Danisco under the name Lactodan B30 or Rylo LA30.

The fatty acid esters of sugars (3) that may be used as nonionic surfactant above may preferably be solid at a temperature of less than or equal to 45° C. and may be chosen in particular from the group comprising esters or mixtures of esters of C₈-C₂₂ fatty acids and of sucrose, maltose, glucose or fructose, and esters or mixtures of esters of C₁₄-C₂₂ fatty acids and of methylglucose.

The C₈-C₂₂ or C₁₄-C₂₂ fatty acids forming the fatty acid unit of the esters that may be used in the present invention comprise a saturated or unsaturated linear alkyl chain, of 8 to 22 or of 14 to 22 carbon atoms, respectively. The fatty acid unit of the esters may be chosen in particular from stearates, behenates, arachidonates, palm itates, myristates, laurates and caprates, and mixtures thereof. Stearates are preferably used.

As examples of esters or mixtures of esters of fatty acid and of sucrose, maltose, glucose or fructose, mention may be made of sucrose monostearate, sucrose distearate and sucrose tristearate and mixtures thereof, such as the products sold by the company Croda under the name Crodesta F50, F70, F110 and F160; and an example of esters or mixtures of esters of fatty acid and of methylglucose that may be mentioned is methylglucose-polyglyceryl-3 distearate, sold by the company Goldschmidt under the name Tego-Care 450. Mention may also be made of glucose or maltose monoesters such as methyl-o-hexadecanoyl-6-D-glucoside and o-hexadecanoyl-6-D-maltoside.

The fatty alcohol ethers of sugars (3) that may be used as nonionic surfactant above may be solid at a temperature of less than or equal to 45° C. and may be chosen in particular from the group comprising ethers or mixtures of ethers of C₈-C₂₂ fatty alcohols and of glucose, maltose, sucrose or fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty acid unit of the ethers that may be used in the nanoemulsion of the invention comprise a saturated or unsaturated linear alkyl chain containing, respectively, from 8 to 22 or from 14 to 22 carbon atoms. The fatty acid unit of the ethers may be chosen in particular from decyl, cetyl, behenyl, arachidyl, stearyl, palm ityl, myristyl, lauryl, capryl and hexadecanoyl units, and mixtures thereof such as cetearyl.

As examples of fatty alkyl ethers of sugars, mention may be made of alkylpolyglucosides such as decyl glucoside and lauryl glucoside sold, for example, by the company Henkel under the respective names Plantaren 2000 and Plantaren 1200, cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Henkel, and also arachidyl glucoside, for example in the form of a mixture of arachidyl alcohol and behenyl alcohol and arachidyl glucoside, sold under the name Montanov 202 by the company SEPPIC.

The surfactant used is more particularly sucrose monostearate, sucrose distearate or sucrose tristearate and mixtures thereof, methyl glucose-polyglyceryl-3 distearate and alkylpolyglucosides.

The fatty acid esters of glycerol (4) that may be used as nonionic surfactant above, which are solid at a temperature of less than or equal to 45° C., may be chosen in particular from the group comprising esters formed from at least one acid comprising a saturated linear alkyl chain containing from 12 to 22 carbon atoms and from 1 to 12 glycerol units. One or more of these fatty acid esters of glycerol may be used in the present invention.

These esters may be chosen in particular from glyceryl stearates, behenates, arachidates and palm itates, and mixtures thereof. Glyceryl stearates and palm itates are preferably used.

As examples of surfactants that may be used in the present invention, mention may be made of decaglyceryl monostearate, distearate, tristearate and pentastearate (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1 S, 2 S, 3 S and 5 S by the company Nikko, and diglyceryl monostearate (CTFA name: polyglyceryl-2 stearate), such as the product sold by the company Nikko under the name Nikkol DGMS.

The fatty acid esters of sorbitan (4) that may be used as nonionic surfactant above, which are solid at a temperature of less than or equal to 45° C., may be chosen from the group comprising esters of C₁₆-C₂₂ fatty acid and of sorbitan and oxyethylenated esters of C₁₆-C₂₂ fatty acid and of sorbitan. They are formed from at least one fatty acid comprising at least one saturated linear alkyl chain containing, respectively, from 16 to 22 carbon atoms and from sorbitol or ethoxylated sorbitol. The oxyethylenated esters generally comprise from 1 to 100 ethylene glycol units and preferably from 2 to 40 ethylene oxide (EO) units.

These esters may be chosen in particular from stearates, behenates, arachidates and palm itates, and mixtures thereof. Stearates and palm itates are preferably used.

As examples of the above nonionic surfactant that may be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The ethoxylated fatty acid ethers (4) that are solid at a temperature of less than or equal to 45° C., which may be used as nonionic surfactant above, are preferably ethers formed from 1 to 100 ethylene oxide units and from at least one fatty alcohol chain containing from 16 to 22 carbon atoms. The fatty chain of the ethers may be chosen in particular from behenyl, arachidyl, stearyl and cetyl units, and mixtures thereof, such as cetearyl. Examples of ethoxylated fatty acid ethers that may be mentioned are behenyl alcohol ethers comprising 5, 10, 20 and 30 ethylene oxide units (CTFA names: beheneth-5, beheneth-10, beheneth-20, beheneth-30), such as the products sold under the names Nikkol BBS, BB10, BB20 and BB30 by the company Nikko, and stearyl alcohol ether comprising 2 ethylene oxide units (CTFA name: steareth-2), such as the product sold under the name Brij 72 by the company ICI.

The ethoxylated fatty acid esters (4) that are solid at a temperature of less than or equal to 45° C., which may be used as nonionic surfactant above, are esters formed from 1 to 100 ethylene oxide units and from at least one fatty acid chain containing from 16 to 22 carbon atoms. The fatty chain in the esters may be chosen in particular from stearate, behenate, arachidate and palmitate units, and mixtures thereof. Examples of ethoxylated fatty acid esters that may be mentioned are the stearic acid ester comprising 40 ethylene oxide units, such as the product sold under the name Myrj 52 (CTFA name: PEG-40 stearate) by the company ICI, and also the behenic acid ester comprising 8 ethylene oxide units (CTFA name: PEG-8 behenate), such as the product sold under the name Compritol HD5 ATO by the company Gattefossé.

The block copolymers of ethylene oxide (A) and of propylene oxide (B) (5) that may be used as surfactants in the nanoemulsion according to the invention may be chosen in particular from the block copolymers of formula (IV):

HO(C₂H₄O)ₓ(C₃H₆O)_{y}(C₂H₄O)_{z}H (IV)

in which x, y and z are integers such that x+z is in the range from 2 to 100 and y is in the range from 14 to 60, and mixtures thereof, and more particularly from the block copolymers of formula (IV) with an HLB value in the range from 8.0 to 14.0.

As silicone surfactants (6) that may be used according to the present invention, mention may be made of those described in documents U.S. Pat. No. 5,364,633 and U.S. Pat. No. 5,411,744.

The silicone surfactant (6) as nonionic surfactant above may preferably be a compound of formula (I):

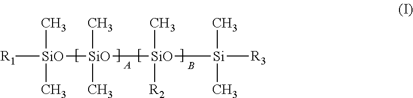

in which:

R₁, R₂ and R₃, independently of each other, represent a C₁-C₆ aryl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;

A is an integer in the range from 0 to 200;

B is an integer in the range from 0 to 50; on condition that A and B are not simultaneously equal to zero;

x is an integer in the range from 1 to 6;

y is an integer in the range from 1 to 30;

z is an integer in the range from 0 to 5.

According to a preferred embodiment of the invention, in the compound of formula (I), the alkyl radical is a methyl radical, x is an integer in the range from 2 to 6 and y is an integer in the range from 4 to 30.

As examples of silicone surfactants of formula (I), mention may be made of the compounds of formula (II):

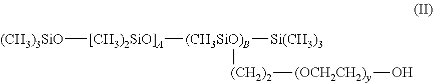

in which A is an integer in the range from 20 to 105, B is an integer in the range from 2 to 10 and y is an integer in the range from 10 to 20.

As examples of silicone surfactants of formula (I), mention may also be made of the compounds of formula (III):

H—(OCH₂CH₂)_{y}—(CH₂)₃—[(CH₃)₂SiO]_{A}—(CH₂)₃—(OCH₂CH₂)_{y}—OH (III)

in which A′ and y are integers in the range from 10 to 20.

The compounds of the invention that may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

It is preferable for the nonionic surfactant (b) with an HLB value of from 8.0 to 14.0, preferably from 9.0 to 13.5 and more preferably from 10.0 to 13.0 to be chosen from:
polyethylene glycol isostearate or oleate (8 to 10 mol of ethylene oxide),
polyethylene glycol isocetyl, behenyl ether or isostearyl ether (8 to 10 mol of ethylene oxide),
polyglyceryl monolaurate or dilaurate comprising 3 to 6 glycerol units,
polyglyceryl mono(iso)stearate comprising 3 to 6 glycerol units,
polyglyceryl monooleate comprising 3 to 6 glycerol units, and
polyglyceryl dioleate comprising 3 to 6 glycerol units.

According to a preferred embodiment of the present invention, the nonionic surfactant with an HLB value from 8.0 to 14.0, preferably from 9.0 to 13.5 and more preferably from 10.0 to 13.0 is chosen from polyglyceryl fatty acid esters and monooxyethylene or polyoxyethylene fatty acid esters.

It is preferable for the polyglyceryl fatty acid ester to comprise esters of a fatty acid and of polyglycerol containing 70% or more of polyglycerol in which the degree of polymerization is 4 or more, preferably esters of a fatty acid and of polyglycerol containing an amount greater than or equal to 60% of polyglycerol in which the degree of polymerization is between 4 and 11, and more preferably esters of a fatty acid and of polyglycerol containing an amount greater than or equal to 30% of polyglycerol in which the degree of polymerization is 5.

The polyglyceryl fatty acid ester may be chosen from monoesters, diesters and triesters of a saturated or unsaturated acid, preferably a saturated acid, comprising 2 to 30 carbon atoms, preferably 6 to 30 carbon atoms and more preferably 8 to 30 carbon atoms, such as lauric acid, oleic acid, stearic acid, isostearic acid, capric acid, caprylic acid and myristic acid.

It is preferable for the polyglyceryl fatty acid ester to be chosen from the group consisting of PG-4 laurate, PG-5 laurate, PG-5 dilaurate, PG-5 oleate, PG-5 dioleate, PG-6 tricaprylate, PG-5 myristate, PG-5 trimyristate, PG-5 stearate, PG-5 isostearate, PG-5 trioleate, PG-6 caprylate and PG-6 tricaprylate.

It is preferable for the monooxyethylene or polyoxyethylene fatty acid ester to contain a (poly)oxyalkylene fragment derived from 1 to 20 oxyalkylenes, preferably from 3 to 15 oxyalkylenes and more preferably from 8 to 10 oxyalkylenes.

The oxyalkylene fragment may be derived from alkylene glycols such as ethylene glycol, propylene glycol, butylene glycol, pentylene glycol, hexylene glycol, and the like. The oxyalkylene fragment may contain a number of moles of ethylene oxide and/or propylene oxide of between 1 and 100 and preferably between 2 and 50. Advantageously, the nonionic surfactants do not comprise any oxypropylene units.

The monooxyethylene or polyoxyethylene fatty acid ester may be chosen from monoesters and diesters of a saturated or unsaturated acid, preferably a saturated acid, comprising from 2 to 30 carbon atoms, preferably from 6 to 30 carbon atoms and more preferably from 8 to 30 carbon atoms, such as lauric acid, oleic acid, stearic acid, isostearic acid, capric acid, caprylic acid and myristic acid.

Examples of monooxyethylene or polyoxyethylene fatty acid esters that may be mentioned comprise a linear or branched, saturated or unsaturated C₂-C₃₀, preferably C₆-C₃₀ and more preferably C₈-C₂₂ acid esters of polyethylene glycols.

Examples of monooxyethylene or polyoxyethylene fatty acid esters that may be mentioned comprise adducts of ethylene oxide with lauric acid, myristic acid, palm itic acid, stearic acid, isostearic acid, oleic acid or behenic acid esters, and mixtures thereof, in particular those containing from 8 to 30 oxyethylene groups, such as PEG-8 to PEG-30 laurate (under the CTFA names: PEG-8 laurate to PEG-30 laurate); PEG-8 to PEG-30 myristate (under the CTFA names: PEG-8 myristate to PEG-30 myristate); PEG-8 to PEG-30 palmitate (under the CTFA names: PEG-8 palmitate to PEG-30 palmitate); PEG-8 to PEG-30 stearate (under the CTFA names: PEG-8 stearate to PEG-30 stearate); PEG-8 to PEG-30 isostearate (under the CTFA names: PEG-8 isostearate to PEG-30 isostearate); PEG-8 to PEG-30 oleate (under the CTFA names: PEG-8 oleate to PEG-30 oleate); PEG-8 to PEG-30 behenate (under the CTFA names: PEG-8 behenate to PEG-30 behenate); and mixtures thereof.

It is preferable for the fatty acid ester of polyglycol to be chosen from the group consisting of PEG-8 isostearate, PEG-8 stearate, PEG-10 isostearate, PEG-10 oleate, PEG-10 isocetyl ether, PEG-10 behenyl ether and PEG-10 isostearyl ether, and a mixture thereof.

The amount of the nonionic surfactant (b) with an HLB value of from 8.0 to 14.0, preferably from 9.0 to 13.5 and more preferably from 10.0 to 13.0 in the cosmetic composition according to the present invention is not limited, and may be in the range from 0.1% to 30% by weight, preferably from 1% to 25% by weight and more preferably from 3% to 20% by weight relative to the total weight of the composition.

[C-glycoside Compound]

The cosmetic composition according to the present invention comprises at least one C-glycoside compound.

The C-glycosides are preferably of general formula (I) below:

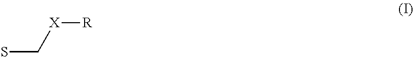

in which:
R represents a saturated or unsaturated C₁-C₁₀ and in particular C₁-C₄ alkyl radical, which may be optionally substituted with at least one radical chosen from OH, COOH, Y and COOR″₂ with R″₂ being a saturated C₁-C₄ alkyl radical, Y denotes a phenyl radical or a heterocycle, optionally substituted with 1 to 5 (ORₐ) groups,
   S represents a monosaccharide or a polysaccharide comprising up to 20 sugar units, in particular up to 6 sugar units, in pyranose and/or furanose form and of the L and/or D series, it being possible for said monosaccharide or polysaccharide to be substituted with a hydroxyl group which must be free, and optionally with one or more optionally protected amine functions, and

X represents a radical chosen from the following groups: —CO—, —CH(OR′)—, —CH (NH₂)—, CHNR_{b}R_{c}; CHNHOR_{d}, —C(OR′)—, —C(NH₂—, CNR_{b}R_{c}; CNHOR_{d} —CH(NHCH₂CH₂CH₂OH)—, —CH(NHPh)— and —CH(CH₃)— and in particular a radical —CO—, —CH(OH)— or —CH(NH₂)— and more particularly a radical —CH(OH)—,
R′ denotes:
a hydrogen atom;
a saturated linear C1-C18 alkyl radical,
an unsaturated linear C2-C18 alkyl radical,
a saturated or unsaturated branched C3-C18 alkyl radical,
a saturated or unsaturated C5 or C6 cyclic radical,
a linear or branched, saturated or unsaturated C2-C18, or saturated or unsaturated C5 or C6 cyclic acyl radical.
Rₐ denotes:
   a hydrogen atom
   a linear or branched C1-C4 alkyl radical, or a linear or branched unsaturated C3-C4 hydrocarbon-based radical
   a linear or branched C2-C18 acyl or linear or branched C2-C18 alkenylcarbonyl radical
R_{b} denotes:
   a hydrogen atom
   a linear C2-C18 or branched C3-C18 alkyl radical, or a linear or branched unsaturated C3-C4 hydrocarbon-based radical or a radical —CH(Z₁)—CO₂Z₂ in which Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT,

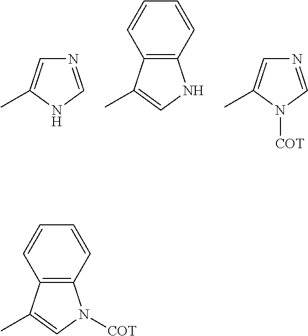

and/or interrupted with a group —NH—, —N—(COT)— or —S— with T denoting a linear or branched C1-C6 or cyclic C3-C6 alkyl radical.

and Z₂ denotes a hydrogen atom or a linear or branched C1-C6 alkyl radical

R_{c} denotes:
a hydrogen atom
a linear C1-C4 or branched C3-C4 alkyl radical, or a linear or branched unsaturated C3-C4 hydrocarbon-based radical, the said radical being optionally substituted with a phenyl group.

Rd denotes:
a hydrogen atom
a linear C1-C18 or branched C3-C18 alkyl radical, or a linear or branched unsaturated C3-C18 hydrocarbon-based radical, the said radical being optionally substituted with a phenyl group,

the bond S—CH₂—X represents a bond of C-anomeric nature, which may be α or β,

and also the cosmetically acceptable salts thereof, solvates thereof such as hydrates, and optical and geometrical isomers thereof.

The C-glycosides of formula (I) that are useful for performing the invention are in particular those for which R denotes a saturated linear C₁-C₆, in particular C₁-C₄ and preferentially C₁-C₂ alkyl radical and more preferentially a methyl radical. Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl and cyclohexyl groups.

According to one embodiment of the invention, a C-glycoside compound may be used corresponding to formula (I) for which S may represent a monosaccharide or a polysaccharide comprising up to 6 sugar units, in pyranose and/or furanose form and of L and/or D series, the said mono- or polysaccharide containing at least one mandatorily free hydroxyl function and/or optionally one or more mandatorily protected amine functions, X and R otherwise conserving all the definitions given previously. Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose which may be advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, and especially D-xylose.

The acceptable salts of the compounds described in the present invention include conventional non-toxic salts of the said compounds, such as those formed from organic or mineral acids. By way of example, mention may be made of the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also include one or more heteroatoms chosen from O and N, for example in the form of hydroxyl groups. Mention may be made especially of propionic acid, acetic acid, terephthalic acid, citric acid and tartaric acid.

When the compound of formula (I) comprises an acidic group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine, 2-dimethylaminopropanol and 2-amino-2-(hydroxymethyl)-1,3-propanediol. Mention may also be made of lysine or 3-(dimethylam ino)propylamine.

The solvates that are acceptable for the compounds described in the present invention comprise conventional solvates such as those formed during the final step of preparation of the said compounds due to the presence of solvents. Mention may be made, by way of example, of the solvates due to the presence of water or of linear or branched alcohols, such as ethanol or isopropanol.

According to a first embodiment, preferentially, use is made of a C-glycoside derivative of formula (I) in which:
R denotes an unsubstituted linear C₁-C₄ alkyl radical, especially C₁-C₂, in particular methyl;
S represents a monosaccharide as described previously, chosen in particular from D-glucose, D-xylose, N-acetyl-D-glucosamine and L-fucose, and in particular D-xylose;
X represents a group chosen from —CO—, —CH(OH)— and —CH(NH₂)— and preferentially a group —CH(OH)—.

As non-limiting illustrations of the C-glycoside compounds that are more particularly suitable for use in the invention, mention may be made especially of the following compounds:
C-β-D-xylopyranoside-n-propane-2-one,
C-α-D-xylopyranoside-n-propan-2-one,

C-β-D-xylopyranoside-2-hydroxypropane,

C-α-D-xylopyranoside-2-hydroxypropane,

1-(C-β-D-fucopyranoside)propan-2-one,

1-(C-α-D-fucopyranoside)propan-2-one,

1-(C-β-L-fucopyranoside)propan-2-one,

1-(C-α-L-fucopyranoside)propan-2-one,

1-(C-β-D-fucopyranoside)-2-hydroxypropane,

1-(C-α-D-fucopyranoside)-2-hydroxypropane,

1-(C-β-3-L-fucopyranoside)-2-hydroxypropane,

1-(C-α-L-fucopyranoside)-2-hydroxypropane,

1-(C-β-D-glucopyranosyl)-2-hydroxypropane,

1-(C-α-D-glucopyranosyl)-2-hydroxypropane,

1-(C-β-D-galactopyranosyl)-2-hydroxypropane,

1-(C-α-D-galactopyranosyl)-2-hydroxypropane,

1-(C-β-D-fucofuranosyl)propan-2-one,

1-(C-α-D-fucofuranosyl)propan-2-one,

1-(C-β-L-fucofuranosyl)propan-2-one,

1-(C-α-L-fucofuranosyl)propan-2-one,

C-β-D-maltopyranoside-n-propane-2-one,

C-α-D-maltopyranoside-n-propan-2-one,

C-β-D-maltopyranoside-2-hydroxypropane,

C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and preferably C-β-D-xylopyranoside-2-hydroxypropane, may be advantageously used for the preparation of a composition according to the invention.

According to a particular embodiment, the C-glycoside compound may be C-β-D-xylopyranoside-2-hydroxypropane (or hydroxypropyltetrahydropyrantriol) in the form of a solution containing 30% by weight of active material in a water/propylene glycol mixture (60%/40% by weight).

A C-glycoside derivative that is suitable for use in the invention may especially be obtained via the synthetic method described in document WO 02/051 828.

According to a second embodiment, the C-glycosides are preferably chosen from the C-glycosides of general formula (I) in which S is xylose and R represents a saturated or unsaturated C2 alkyl radical substituted with a radical Y which denotes a phenyl radical or a heterocycle, optionally substituted with 1 to 5 groups (ORₐ) and represented by formula (II) below:

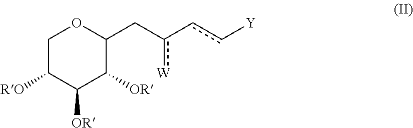

in which:
the compounds of formula (I) are xylose derivatives
Y denotes a phenyl radical or a heterocycle, optionally substituted with 1 to 5 groups (ORₐ)
W═—OR′; (═O ); NR_{b}R_{c}; NHOR_{d}
R′ denotes:
a hydrogen atom;
a saturated linear C1-C18 alkyl radical,
an unsaturated linear C2-C18 alkyl radical,
a saturated or unsaturated branched C3-C18 alkyl radical,
a saturated or unsaturated C5 or C6 cyclic radical,
a linear or branched, saturated or unsaturated C2-C18, or saturated or unsaturated C5 or C6 cyclic acyl radical.

Rₐ denotes:
a hydrogen atom
a linear or branched C1-C4 alkyl radical, or a linear or branched unsaturated C3-C4 hydrocarbon-based radical
a linear or branched C2-C18 acyl or linear or branched C2-C18 alkenylcarbonyl radical

When Y denotes a phenyl radical or a heterocycle substituted with 2 to 5 groups (ORₐ), two adjacent groups ORₐ may together form a divalent radical —O—CH₂—O with the proviso that when W═OH, the compound does not comprise an ethylenic double bond alpha to the carbon bearing this OH.

R_{b} denotes:
a hydrogen atom
a linear C2-C18 or branched C3-C18 alkyl radical, or a linear or branched unsaturated C3-C4 hydrocarbon-based radical or a radical —CH(Z₁)—CO₂Z₂ in which
   Z₁ denotes a hydrogen atom or a linear or branched C1-C6, or saturated or unsaturated C3-C6 cyclic alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT,

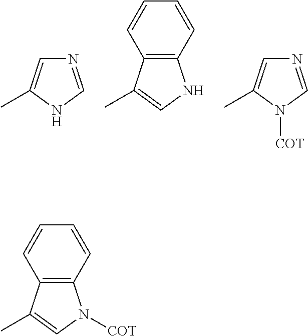

and/or interrupted with a group —NH—, —N—(COT)— or —S— with T denoting a linear or branched C1-C6 or cyclic C3-C6 alkyl radical.

and Z₂ denotes a hydrogen atom or a linear or branched C1-C6 alkyl radical

R_{c} denotes:
a hydrogen atom
a linear C1-C4 or branched C3-C4 alkyl radical, or a linear or branched unsaturated C3-C4 hydrocarbon-based radical, the said radical being optionally substituted with a phenyl group.

Rd denotes:
a hydrogen atom
a linear C1-C18 or branched C3-C18 alkyl radical, or a linear or branched unsaturated C3-C18 hydrocarbon-based radical, the said radical being optionally substituted with a phenyl group,
   and also the cosmetically acceptable salts thereof, solvates thereof such as hydrates, and stereoisomers thereof.

For the purposes of the present invention, the term “heterocycle” denotes a saturated or unsaturated 5- to 10-membered cyclic hydrocarbon-based radical, including an aromatic radical, comprising at least one heteroatom chosen from O, S and N. Preferably, heterocycle denotes a pyridine, pyrimidine or indole radical and more preferentially pyridine or indole.

The preferred compounds of formula (II) are those for which:
the compounds of formula (II) are xylose derivatives
Y denotes a phenyl radical or a heterocycle, optionally substituted with 1 to 5 groups (ORₐ)
W═—OR′; (═O); NR_{b}R_{c}; NHOR_{d}
R′ denotes:
a hydrogen atom,
a linear or branched, saturated or unsaturated C1-C12, or saturated cyclic C5 or C6 alkyl radical,
a linear or branched, saturated or unsaturated C1-C6, or saturated cyclic C5 or C6 acyl radical,

Rₐ denotes:
a hydrogen atom
a linear or branched C1-C4 alkyl radical,
a linear or branched C1-C6 acyl radical,
   When Y denotes a phenyl radical or a heterocycle substituted with 2 to 5 groups (ORₐ), two adjacent groups ORₐ may together form a divalent radical ‘3O—CH₂—O with the proviso that when W═OH, the compound does not comprise an ethylenic double bond alpha to the carbon bearing this OH.

R_{b} denotes:
a hydrogen atom
a linear C2-C12 or branched C3-C12 alkyl radical, or a radical —CH(Z₁)—CO₂Z₂ in which
   Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT,

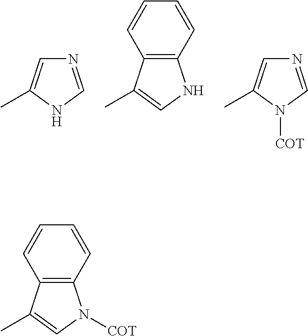

and/or interrupted with a group —NH—, —N—(COT)— or —S— with T denoting a linear C1-C6 or cyclic C5-C6 alkyl radical,
and Z₂ denotes a hydrogen atom or a linear C1-C6 alkyl radical

R_{c} denotes:
a hydrogen atom
a linear C1-C4 or branched C3-C4 alkyl radical, or a linear or branched unsaturated C3-C4 hydrocarbon-based radical, the said radical being optionally substituted with a phenyl group.

Rd denotes:
a hydrogen atom
a linear C1-C12 or branched C3-C12 alkyl radical, the said radical being optionally substituted with a phenyl group,
   and also the cosmetically acceptable salts thereof, solvates thereof such as hydrates, and stereoisomers thereof.

For the purposes of the present invention, the term “heterocycle” denotes a saturated or unsaturated 5- to 10-membered cyclic hydrocarbon-based radical, including an aromatic radical, comprising at least one heteroatom chosen from O, S and N. Preferably, heterocycle denotes a pyridine, pyrimidine or indole radical and more preferentially pyridine or indole.

The compounds of formula (II) that are particularly preferred are those for which:
the compounds of formula (II) are xylose derivatives
Y denotes a phenyl radical or a heterocycle, optionally substituted with 1 to 3 groups (ORₐ)
W═—OR′; (═O); NR_{b}R_{c}; NHOR_{d}
R′ denotes:
a hydrogen atom,
a linear or branched, saturated or unsaturated C1-C4 alkyl radical,
a linear or branched C1-C6 acyl radical.

Rₐ denotes:
a hydrogen atom
a linear or branched C1-C4 alkyl radical,
a linear or branched C1-C6 acyl radical,
   when Y denotes a phenyl radical or a heterocycle substituted with 2 or 3 groups (ORₐ), two adjacent groups ORₐ may together form a divalent radical —O—CH₂—O

with the proviso that when W═OH, the compound does not comprise an ethylenic double bond alpha to the carbon bearing this OH.

R_{b} denotes:
a hydrogen atom
a linear C2-C8 or branched C3-C8 alkyl radical, or a radical —CH(Z₁)—CO₂Z₂ in which Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT,

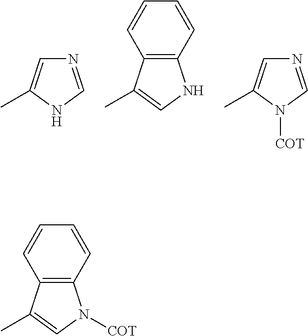

and/or interrupted with a group —NH—, —N—(COT)— or —S— with T denoting a linear C1-C6 or cyclic C5-C6 alkyl radical,

and Z₂ denotes a hydrogen atom or a linear C1-C6 alkyl radical

R_{c} denotes:
a hydrogen atom
a linear C1-C4 or branched C3-C4 alkyl radical, optionally substituted with a phenyl group.

Rd denotes:
a hydrogen atom
a linear C1-C4 or branched C3-C4 alkyl radical, the said radical being optionally substituted with a phenyl group,
   and also the cosmetically acceptable salts thereof, solvates thereof such as hydrates, and stereoisomers thereof.

For the purposes of the present invention, the term “heterocycle” denotes a saturated or unsaturated 5- to 10-membered cyclic hydrocarbon-based radical, including an aromatic radical, comprising at least one heteroatom chosen from O, S and N.

Preferably, heterocycle denotes a pyridine, pyrimidine or indole radical and more preferentially pyridine or indole.

The compounds of formula (II) that are more particularly preferred are the following:

Compound 1.:

### (3R,4S, 5R)-2-[2-hydroxy-4-(4-hydroxy-3-methoxyphenyl)butyl]tetrahydro-2H-pyran-3,4,5-triol

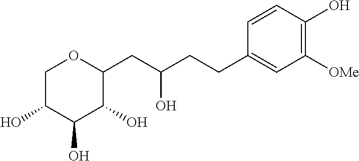 [SMILES: COc1cc(CCC(O)CC2OC[C@@H](O)[C@H](O)[C@H]2O)ccc1O]

Compound 2.

### 4-(4-hydroxy-3-methoxyphenyl)-1-[(3R,4S,5R)-3,4,5-trihydroxytetrahydro-2H-pyran-2-yl]butan-2-one

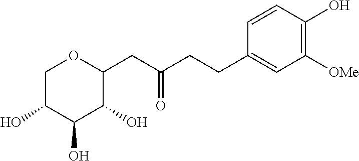 [SMILES: COc1cc(CCC(=O)CC2OC[C@@H](O)[C@H](O)[C@H]2O)ccc1O]

Compound 3.

### (3R,4S, 5R)-2-[2-hydroxy-4-(4-hydroxyphenyl)butyl]tetrahydro-2H-pyran-3,4,5-triol

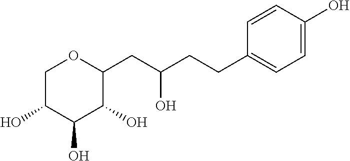 [SMILES: Oc1ccc(CCC(O)CC2OC[C@@H](O)[C@H](O)[C@H]2O)cc1]

Compound 4.

### 4-(4-hydroxyphenyl)-1-R3R,4S,5R)-3,4,5-trihydroxytetrahydro-2H-pyran-2-yl]butan-2-one

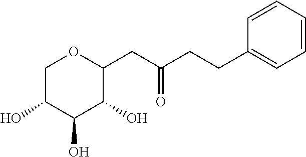 [SMILES: O=C(CCc1ccccc1)CC1OC[C@@H](O)[C@H](O)[C@H]1O]

Compound 5.

### (3R,4S,5R)-2-[2-(benzylamino)-4-(4-hydroxy-3-methoxyphenyl)butyl]tetrahydro-2H-pyran-3,4,5-triol

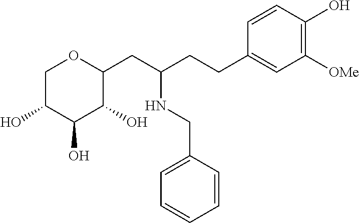 [SMILES: COc1cc(CCC(CC2OC[C@@H](O)[C@H](O)[C@H]2O)NCc2ccccc2)ccc1O]

Compound 6.

### ethyl

### {[3-(4-hydroxy-3-methoxyphenyl)-1-{[(3R,4S,5R)-3,4,5-trihydroxytetrahydro-2H-pyran-2-yl]methyl}propyl]amino}(phenyl)acetate

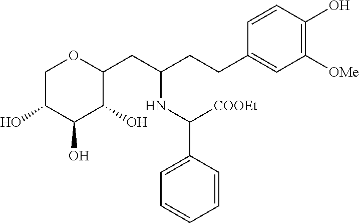 [SMILES: CCOC(=O)C(NC(CCc1ccc(O)c(OC)c1)CC1OC[C@@H](O)[C@H](O)[C@H]1O)c1ccccc1]

Compound 7.

### (3E)-4-phenyl-1-[(3R,4S,5R)-3,4,5-trihydroxytetrahydro-2H-pyran-2-yl]but-3-en-2-one

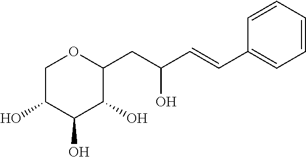 [SMILES: OC(/C=C/c1ccccc1)CC1OC[C@@H](O)[C@H](O)[C@H]1O]

Compound 8.

### (3E)-4-(4-hydroxy-3,5-dimethoxyphenyl)-1-[(3R,4S,5R)-3,4,5-trihydroxytetrahydro-2H-pyran-2-yl]but-3-en-2-one

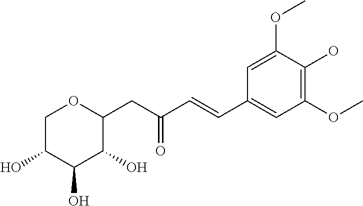 [SMILES: COc1cc(/C=C/C(=O)CC2OC[C@@H](O)[C@H](O)[C@H]2O)cc(OC)c1O]

Compound 9.

### (3R,4S, 5R)-2-[2-hydroxy-4-(2-hydroxyphenyl)butyl]tetrahydro-2H-pyran-3,4,5-triol

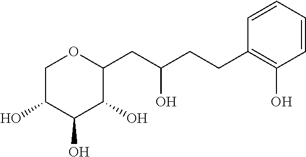 [SMILES: Oc1ccccc1CCC(O)CC1OC[C@@H](O)[C@H](O)[C@H]1O]

Compound 10:

### (3R,4S, 5R)-2-[2-hydroxy-4-(3-hydroxy-4-methoxyphenyl)butyl]tetrahydro-2 H-pyran-3,4,5-triol

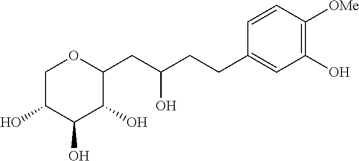 [SMILES: COc1ccc(CCC(O)CC2OC[C@@H](O)[C@H](O)[C@H]2O)cc1O]

Compound 11.

### (3R,4S,5R)-2-[2-hydroxy-4-(2,4-di-hydroxyphenyl)butyl]tetrahydro-2H-pyran-3,4,5-triol

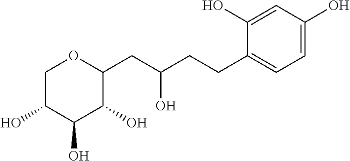 [SMILES: Oc1ccc(CCC(O)CC2OC[C@@H](O)[C@H](O)[C@H]2O)c(O)c1]

Compound 12.

### (3R,4S,5R)-2-[2-hydroxy-4-(3-ethoxy-4-hydroxyphenyl)butyl]tetrahydro-2H-pyran-3,4,5-triol

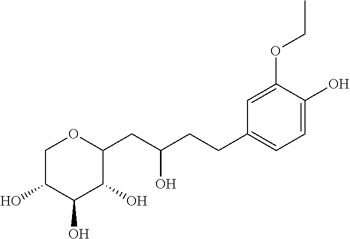 [SMILES: CCOc1cc(CCC(O)CC2OC[C@@H](O)[C@H](O)[C@H]2O)ccc1O]

Compound 13.

### 5,9-anhydro-1,2,4-trideoxy-1-pyridin-3-yl-D-xylononitol

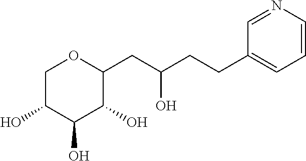 [SMILES: OC(CCc1cccnc1)CC1OC[C@@H](O)[C@H](O)[C@H]1O]

Compound 14.

### (3R,4S,5R)-2-[(2E)-2-(methoxyimino)-4-phenylbutyl]tetrahydro-2H-pyran-3,4,5-triol

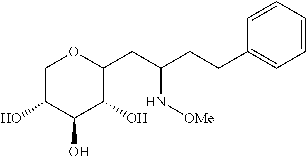 [SMILES: CONC(CCc1ccccc1)CC1OC[C@@H](O)[C@H](O)[C@H]1O]

Compound 15.

### 5,9-anhydro-1,2,4-trideoxy-7-O-pentanoyl-1-phenyl-D-xylonon-3-ulose

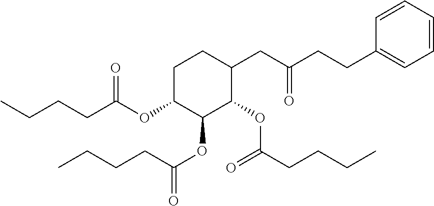 [SMILES: CCCCC(=O)O[C@H]1[C@H](OC(=O)CCCC)CCC(CC(=O)CCc2ccccc2)[C@@H]1OC(=O)CCCC]

Compound 16.

### 5,9-anhydro-1,2,4-trideoxy-1-(3,4,5-trimethoxyphenyl)-D-xylononitol

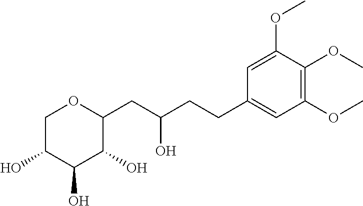 [SMILES: COc1cc(CCC(O)CC2OC[C@@H](O)[C@H](O)[C@H]2O)cc(OC)c1OC]

The C-glycoside compound (c) of formula (II) is preferably chosen from compounds (1) and (11).

According to the invention, the C-glycoside compound (c) is preferably chosen from C-β-D-xylopyranoside-2-hydroxypropane, -α-D-xylopyranoside-2-hydroxypropane, compounds (1) and (11) and more particularly from C-β-D-xylopyranoside-2-hydroxypropane and compound 1.

The compounds of formula (I) and/or (II) may be synthesized according to the general procedure given in patent application EP 2 376 510 or according to patent application FR 1 262 731.

Needless to say, according to the invention, a C-glycoside derivative corresponding to formula (I) and/or (II) may be used alone or as a mixture with other C-glycoside derivatives and in any proportion.

The amount of the C-glycoside compound (c) in the cosmetic composition according to the present invention may range from 0.01% to 20% by weight, preferably from 0.1% to 15% by weight and more preferably from 1% to 10% by weight relative to the total weight of the composition.

[Water]

The cosmetic composition according to the present invention comprises water.

The amount of water is not limited, and may range from 50% to 99% by weight, preferably from 55% to 95% by weight and more preferably from 60% to 90% by weight relative to the total weight of the composition.

[Additional Surfactant]

The cosmetic composition according to the present invention may also comprise at least one nonionic surfactant other than (b) above and/or at least one additional ionic surfactant.

As additional surfactant, use may be made of at least one nonionic surfactant other than b), in particular with an HLB value of less than 8.0 or more than 14.0.

As additional nonionic surfactant, mention may be made of compounds of the type listed in (b) above except that the additional nonionic surfactant has an HLB value of less than 8.0, preferably less than 9.0 and more preferably less than 10.0, and greater than 14.0, preferably greater than 13.5 and more preferably greater than 13.0.

As additional surfactant, use may be made of at least one ionic surfactant. The ionic surfactant may be chosen from cationic surfactants, anionic surfactants and amphoteric surfactants, and is more particularly chosen from anionic surfactants.

According to one embodiment, the cationic surfactant that may be used in the compositions of the invention is chosen from quaternary ammonium salts, for example behenyltrimethylammonium chloride, cetyltrimethylammonium chloride, Quaternium-83, Quaternium-87, Quaternium-22, behenylam idopropyl-2,3-dihydroxypropyldimethylammonium chloride, palm itylam idopropyltrimethylammonium chloride and stearam idopropyldimethylamine.

Anionic Surfactant

The anionic surfactants may comprise at least one group chosen from carboxylic, sulfate, sulfonate and phosphate groups. Use may be made of alkyl phosphates, alkyl sulfosuccinates, amino acid derivatives, alkyl sulfates, alkyl ether sulfates, alkylsulfonates, alkyl isethionates, alkyl taurates, alkyl sulfoacetates, alkylpolypeptides and anionic alkylpolyglucoside derivatives, and mixtures thereof, the alkyl groups containing from 8 to 22 carbon atoms.

1) As alkyl phosphates, examples that may be mentioned include monoalkyl phosphates and dialkyl phosphates, such as the lauryl monophosphate sold under the name MAP 20® by Kao Chemicals, the potassium salt of dodecyl phosphate, the mixture of monoesters and diesters (mainly a diester) sold under the name Crafol AP-31® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7

EO-Phosphate Ester® by Condea, the potassium or triethanolamine salt of mono(C₁₂-C₁₃ alkyl) phosphate, sold under the references Arlatone MAP 230K-40® and Arlatone MAP 230T-60® by Uniqema, the potassium lauryl phosphate sold under the name Dermalcare MAP XC-99/09® by Rhodia Chimie, and the potassium cetyl phosphate sold under the name Arlatone MAP 160K by Uniqema.

2) As surfactant comprising at least one carboxylic group, mention may be made of:
alkylamido ether carboxylates (AEC), such as sodium laurylamido ether carboxylate (3 EO), sold under the name Akypo Foam 30® by Kao Chemicals;
alkyl ether carboxylic acid salts, such as the oxyethylenated sodium lauryl ether carboxylate sold under the name Akypo Soft 45 NV® by Kao Chemicals, oxyethylene-oxyethylene-polyoxyethylene and carboxymethyl fatty acids originating from olive oil, sold under the name Olivem 400® by Biologia E Tecnologia, or the oxyethylenated sodium tridecyl ether carboxylate, sold under the name Nikkol ECTD-6NEX® by Nikkol; and
fatty acid salts (soaps) bearing a C₆ to C₂₂ alkyl chain, which are neutralized with an organic or mineral base such as potassium hydroxide, sodium hydroxide, triethanolamine, N-methylglucamine, lysine or arginine.

3) Mention may be made in particular of amino acid derivatives such as:
(C6-C18)alkyl sarcosinates, such as sodium lauroyl sarcosinate, sold under the name Sarkosyl NL 97® by Ciba or sold under the name Oramix L 30® by SEPPIC, sodium myristoyl sarcosinate, sold under the name Nikkol Sarcosinate MN® by Nikkol, or sodium palmitoyl sarcosinate, sold under the name Nikkol Sarcosinate PN® by Nikkol;
(C6-C18)alkyl alaninates, such as sodium N-lauroyl-N-methylamidopropionate, sold under the name Sodium Nikkol Alaninate LN 30® by Nikkol or sold under the name Alanone ALE® by Kawaken, or triethanolamine-N-lauroyl-N-methylalanine, sold under the name Alanone ALTA® by Kawaken;
(C6-C18)alkyl glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto;
(C6-C18)alkyl aspartates, such as the mixture of triethanolamine-N-lauroyl aspartate and triethanolamine-N-myristoyl aspartate, sold under the name Asparack® by Mitsubishi;
(C6-C18) alkyl glycinates, such as sodium N-cocoyl glycinate, sold under the names Amilite GCS-12® and Amilite GCK 12 by Ajinomoto;
(C6-C18)alkyl citrates, such as the citric monoester of oxyethylene-oxyethylene-oxyethylene (9 mol) coconut alcohols, sold under the name Witconol EC 1129 by Goldschmidt; and
(C6-C18)alkyl galacturonates, such as sodium dodecyl-D-galactoside-uronate, sold by Soliance.

4) As (C6-C18) alkyl methyltaurates, mention may be made of the sodium salt of palm oil methyltaurate, sold under the name Hostapon CT Pate® by Clariant; N-acyl-N-methyltaurates such as sodium N-cocoyl-N-methyltaurate, sold under the name Hostapon LT-SF® by Clariant or sold under the name Nikkol CMT-30-T® by Nikkol, or sodium palmitoyl methyltaurate, sold under the name Nikkol PMT® by Nikkol.

5) The anionic derivatives of alkyl polyglucosides may in particular be citrates, tartrates, sulfosuccinates, carbonates and glycerol ethers obtained from alkyl polyglucosides. Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulfosuccinic ester, sold under the name Essai 512 MP® by SEPPIC, or the sodium salt of cocoylpolyglucoside (1,4) citric ester, sold under the name Eucarol AGE-EC® by Cesalpinia.

It is preferable for the amino acid derivatives to be acylglycine derivatives, in particular an acylglycine salt.

The acylglycine derivatives may be chosen from acylglycine salts (or acylglycinates) or glycine salts (or glycinates), and in particular the following:

i) Acylglycinates of formula (I):

R—HNCH₂COOX (I)

in which:
R represents an acyl group R′C═O, with R′ representing a linear or branched, saturated or unsaturated hydrocarbon-based chain, preferably comprising from 10 to 30 carbon atoms, preferably from 12 to 22 carbon atoms, preferably from 14 to 22 carbon atoms and more preferably from 16 to 20 carbon atoms, and
X represents a cation chosen, for example, from ions of alkali metals, such as Na, Li or K, preferably Na or K, ions of alkaline-earth metals such as Mg, and ammonium groups, and mixtures thereof.

The acyl group may be chosen in particular from lauroyl, myristoyl, behenoyl, palmitoyl, stearoyl, isostearoyl, olivoyl, cocoyl and oleoyl groups, and mixtures thereof.

Preferably, R is a cocoyl group.

ii) Glycinates of formula (II) below:

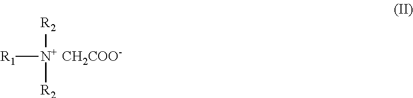

in which:
R₁ represents a linear or branched, saturated or unsaturated hydrocarbon-based chain, comprising from 10 to 30 carbon atoms, preferably from 12 to 22 carbon atoms and more preferably from 16 to 20 carbon atoms; R₁ is advantageously chosen from lauryl, myristyl, palm ityl, stearyl, cetyl, cetearyl and oleyl groups, and mixtures thereof, and preferably stearyl and oleyl groups,
the groups R₂, which are identical or different, represent a group R″OH, R″ being an alkyl group comprising from 2 to 10 carbon atoms and preferably from 2 to 5 carbon atoms.

As compound of formula (I), mention may be made, for example, of the compounds having the INCl name sodium cocoyl glycinate, for instance Amilite GCS-12, sold by Ajinomoto, or potassium cocoyl glycinate, for instance Amilite GCK-12 from Ajinomoto.

As compounds of formula (II), use may be made of dihydroxyethyloleyl glycinate or dihydroxyethylstearyl glycinate.

The amount of the additional surfactant(s) may be from 0.01% by weight to 20% by weight, preferably from 0.10% by weight to 10% by weight and more preferably from 1% by weight to 5% by weight, relative to the total weight of the composition.

[Polyol]

The composition according to the present invention may also comprise at least one polyol.

According to the present invention, the term “polyol” denotes a compound bearing two or more than two hydroxyl groups.

The polyol may be a C₂-C₁₂ polyol, preferably a C₂₋₉ polyol, comprising at least 2 hydroxyl groups and preferably 2 to 5 hydroxyl groups.

The polyol may be a natural or synthetic polyol. The polyol may have a linear, branched or cyclic molecular structure.

The polyol may be chosen from glycerol and derivatives thereof, and glycols and derivatives thereof. The polyol may be chosen from the group consisting of glycerol, diglycerol, polyglycerol, diethylene glycol, propylene glycol, dipropylene glycol, butylene glycol, pentylene glycol, hexylene glycol, 1,3-propanediol, 1,5-pentanediol, polyethylene glycols, especially containing from 5 to 50 ethylene oxide groups, and sugars such as sorbitol.

The polyol may be present in an amount in the range from 0.01% to 30% by weight and preferably from 0.1% to 20% by weight, such as from 1% to 10% by weight, relative to the total weight of the composition.

[Thickener]

The cosmetic composition according to the present invention may also comprise at least one thickener.

The thickener may be chosen from organic and mineral thickeners.

The thickener is preferably chosen from associative thickeners and polysaccharides such as starch and xanthan gum.

In the present context, the term “associative thickener” denotes an amphiphilic thickener comprising both hydrophilic and hydrophobic units, for example comprising at least one C₈-C₃₀ fatty chain and at least one hydrophilic unit.

The viscosity of the cosmetic composition according to the present invention is not particularly limited. The viscosity may be measured at 25° C. with viscometers or rheometers, preferably having cone-plate geometry. Preferably, the viscosity of the cosmetic composition according to the present invention may be, for example, from 1 to 2000 Pa·s and preferably from 1 to 1000 Pa·s at 25° C. and 1 s⁻¹.

The thickener may be present in an amount in the range from 0.001% to 10% by weight and preferably from 0.01% to 10% by weight, for example from 0.1% to 5% by weight, relative to the total weight of the composition.

[Other Components]

The cosmetic composition according to the present invention may also comprise an efficient amount of other components, previously known elsewhere in compositions, especially cosmetic compositions, such as various adjuvants, anti-ageing agents, bleaching agents, anti-greasy skin agents, sequestrants such as EDTA and etidronic acid, UV stabilizers, preserving agents, vitamins or provitamins, for example panthenol, opacifiers, fragrances, plant extracts, cationic polymers, etc.

The cosmetic composition according to the present invention may also comprise at least one organic solvent. Consequently, the organic solvent is preferably water-miscible. Examples of organic solvents that may be mentioned include C₂-C₄ alkanols, such as ethanol and isopropanol; aromatic alcohols such as benzyl alcohol and phenoxyethanol; similar products; and mixtures thereof.

The water-soluble organic solvents may be present in a content ranging from 0 to 20% by weight, preferably from 0.1% to 10% by weight and more preferably from 0.5% to 5% by weight relative to the total weight of the composition.

[Preparation and Properties]

The cosmetic composition according to the present invention may be prepared by mixing the essential and optional components above according to a conventional process. The conventional process comprises mixing with a high-pressure homogenizer (a high-energy process). As a variant, the cosmetic composition may be prepared via a low-energy process such as a phase inversion temperature (PIT) process, a phase inversion concentration (PIC), self-emulsification, and the like. Preferably, the cosmetic composition is prepared via a low-energy process.

The ratio of the nonionic surfactant (b) to the oil (a) may be from 0.25 to 6, preferably from 0.3 to 3 and more preferably from 0.4 to 1.5. In particular, the ratio of the nonionic surfactant (b) to the oil (a) is preferably 1.5 or less, for example from 0.25 to 1.5, preferably from 0.3 to 1.5 and more preferably from 0 to 1.5.

The cosmetic composition according to the present invention is in the form of a nanoemulsion or microemulsion.

The term “microemulsion” may be defined in two ways, i.e. in a broad sense and in a narrower sense. Namely, in one case (“microemulsion in the narrow sense”), the term microemulsion denotes a thermodynamically stable isotropic single liquid phase containing a ternary system having three components comprising an oily component, an aqueous component and a surfactant, and, in the other case (“microemulsion in the broad sense”), among the thermodynamically unstable typical emulsion systems, the term microemulsion also comprises emulsions that have transparent or translucent appearances on account of their smaller particle sizes (Satoshi Tomomasa, et al., Oil Chemistry, vol. 37, No. 11 (1988), pp. 48-53). In the present context, the term “microemulsion” denotes a “microemulsion in the narrow sense”, i.e. a thermodynamically stable isotropic single liquid phase.

The microemulsion denotes a state of a microemulsion of O/W (oil-in-water) type in which the oil is dissolved by micelles, a microemulsion of W/O (water-in-oil) type in which the water is dissolved by inverse micelles, or a bicontinuous microemulsion in which the number of associations of surfactant molecules tends to infinity such that the aqueous phase and the oily phase both have a continuous structure.

The microemulsion may have a dispersed phase with a numerical mean diameter of 100 nm or less, preferably 50 nm or less and more preferably 20 nm or less, measured by laser particle size analysis.

The term “nanoemulsion” presently denotes an emulsion characterized by a dispersed phase with a size of less than 350 nm, the dispersed phase being stabilized by a crown of the nonionic surfactant (b) which may optionally form a liquid crystal phase of lamellar type, at the dispersed phase/continuous phase interface. In the absence of specific opacifiers, the transparency of nanoemulsions is due to the small size of the dispersed phase, this small size being able to be obtained by means of using mechanical energy and in particular a high-pressure homogenizer.

Nanoemulsions may be distinguished from microemulsions by their structure. Specifically, microemulsions are thermodynamically stable dispersions formed, for example, from swollen micelles of nonionic surfactant (b) with the oil (a). Furthermore, microemulsions do not require substantial mechanical energy to be prepared.

The microemulsion may have a dispersed phase with a numerical mean diameter of 300 nm or less, preferably 200 nm or less and more preferably 100 nm or less, measured by laser particle size analysis.

The cosmetic composition according to the present invention may be in the form of an O/W nanoemulsion or microemulsion, a W/O nanoemulsion or microemulsion, or a bicontinuous emulsion. It is preferable for the cosmetic composition according to the present invention to be in the form of an O/W nanoemulsion or microemulsion.

It is preferable for the cosmetic composition according to the present invention to be in the form of an O/W emulsion.

The mean size of the droplets of the oily phase is measured by dynamic light scattering (DLS) with a Vasco particle size analyser.

These measurements are taken on the undiluted emulsion.

The numerical mean size (pm) of the droplets of oily phase of the composition of the invention is less than 300 nm, preferably from 10 nm to 150 nm and more preferably from 20 nm to 100 nm.

The cosmetic composition according to the present invention may have a transparent or slightly translucent appearance, preferably a transparent appearance.

The transparency may be measured by measuring the transmission factor with an absorption spectrometer in the visible region (for example, the transparency is measured with a Hach 2100Q portable turbidimeter at 25° C.). The portable turbidimeter uses the nephelometric principle for measuring turbidity. The nephelometric turbidity measurement depends on the detection of the light scattered by the particles in suspension in the liquid. The measuring unit is the NTU. A 60×25 cm round borosilicate glass tank with a screw stopper is used. The amount of sample required is 15 mL. The measuring range is 0-1000 NTU. The samples are measured undiluted.

The cosmetic composition according to the present invention may preferably have a turbidity of between 1 and 200 NTU and preferably between 5 and 100 NTU.

[Process and Use]

The cosmetic composition according to the present invention may be used for a non-therapeutic process, such as a cosmetic process, for treating the skin, the hair, mucous membranes, the nails, the eyelashes, the eyelids and/or the scalp, by application to the skin, the hair, mucous membranes, the nails, the eyelashes, the eyelids or the scalp.

The present invention also relates to a use of the cosmetic composition according to the present invention, in its native form or in care products and/or washing products and/or makeup products and/or makeup-removing products for bodily and/or facial skin and/or mucous membranes and/or the scalp and/or the hair and/or the nails and/or the eyelashes and/or the eyelids.

The care product may be a lotion, a cream, a hair tonic, a hair conditioner, a sunscreen, and the like. The cleansing product may be a shampoo, a facial cleanser, a hand cleanser, and the like. The makeup product may be a foundation, a mascara, a lipstick, a lip gloss, a face powder, an eyeshadow, a nail varnish, and the like. The makeup-removing product may be a makeup-cleansing product, and the like.

### EXAMPLES

The present invention is described in greater detail by means of examples, which should not, however, be considered as limiting the scope of the present invention.

(Particle size) The particle size is measured using a Vasco-2 machine (Cordouan Technologies) under undiluted conditions.

(Transparency) The transparency is measured using a Hach 2100Q portable turbidimeter at 25° C. A 60×25 cm round borosilicate glass tank with a screw stopper is used. The amount of sample required is 15 mL. The measuring range is 0-1000 NTU. The samples are measured undiluted.

### Example 1 and Comparative Example 2

The following compositions according to Example 1 and Comparative Example 2, described in Table 1, are prepared by mixing the components described in Table 1 as follows: (1) mixing isopropyl myristate and polyglyceryl-5 laurate to form an oily phase; (2) heating the oily phase to about 75° C.; (3) mixing water and (3R,4S, 5R)-2-[2-hydroxy-4-(4-hydroxy-3-methoxyphenyl)butyl]tetrahydro-2H-pyran-3,4,5-triol containing 42.5% active material in propanediol and water; and (4) adding the aqueous phase to the oily phase with stirring to obtain an O/W emulsion.

The numerical values for the amounts of the components described in Table 1 are all based on weight percentages of active materials.

| | | Comparative |
|---|---|---|
| | 1 | 2 |
| Isopropyl myristate | 12 | 12 |
| Octyldodecanol | 1 | 1 |
| Polyglyceryl-2 caprate (Sunsoft Q-10D-C | 4 | 4 |
| from Taiyo Kagaku) | | |
| Polyglyceryl-5 laurate (Sunsoft A-121E-C | 9 | 9 |
| from Taiyo Kagaku) | | |
| (3R,4S,5R)-2-[2-hydroxy-4-(4-hydroxy-3- | 12.3% AM | — |
| methoxyphenyl)butyl]tetrahydro- | | |
| 2H-pyran-3,4,5-triol containing 42.5% active | | |
| material in propanediol and water | | |
| Butylene glycol | 2 | 2 |
| Phenoxyethanol | 0.5 | 0.5 |
| Caprylyl glycol | 0.5 | 0.5 |
| Water qs | 100 | 100 |

The appearance, the oil particle size and the transparency of the O/W emulsions obtained according to Example 1 and Comparative Example 1 are described in Table 2.

### Example 3 and Comparative Example 4

| | | Comparative |
|---|---|---|
| | 3 | 4 |
| Ethylhexyl palmitate | 5 | 5 |
| Polyglyceryl-5 laurate (Sunsoft A-121E-C from | 5 | 5 |
| Taiyo Kagaku) | | |
| Butylene glycol | 20 | 20 |
| Sodium methyl stearoyl taurate | 0.1 | 0.1 |
| (Nikkol SMT from Nikko) | | |
| Phenoxyethanol | 0.5 | 0.5 |
| Caprylyl glycol | 0.5 | 0.5 |
| Hydroxypropyltetrahydropyrantriol | 1% AM | |
| Water qs | 100 | 100 |

As clearly indicated by the results below, it was observed that the cosmetic compositions in the form of an O/W emulsion according to the present invention 1 and 3 show better transparency and are stable.

TABLE 2

| | | | Numerical | |
|---|---|---|---|---|
| | | Mean | mean | |
| | Macroscopic | turbidity | diameter | |
| | appearance | (NTU) | (nm) | Stability |
| 1 | Transparent | 25 | 20 | Stable 2 |
| invention | fluid | | | months |
| | | | | at 45° C. |
| 2 | Opaque white | >1000 | >1000 | 2 |
| Comparative | fluid: 2 phases | | | phases |
| 3 | Transparent | 77 | 30 | Stable 2 |
| Invention | fluid | | | months |
| | | | | at 45° C. |
| 4 | Opaque white | >1000 | >1000 | 2 |
| Comparative | fluid: 2 phases | | | phases |

1. Cosmetic composition in the form of a nanoemulsion or microemulsion, comprising:
(a) at least one oil;
(b) at least one nonionic surfactant with an HLB value from 8.0 to 14.0;
(c) at least one C-glycoside compound
(d) water.

2. Cosmetic composition according to claim 1, in which the oil (a) is chosen from the group consisting of oils of plant origin, mineral oils, synthetic oils, silicone oils and hydrocarbon-based oils.

3. Cosmetic composition according to claim 1, in which the oil (a) is chosen from hydrocarbon-based oils which are in the form of a liquid at room temperature.

4. Cosmetic composition according to claim 1, in which the oil (a) is chosen from oils with a molecular weight of less than 600 g/mol.

5. Cosmetic composition according to claim 1, in which the amount of oil (a) is in the range from 0.1% to 50% by weight relative to the total weight of the composition.

6. Cosmetic composition according to claim 1, in which the nonionic surfactant (b) is chosen from:
surfactants that are fluid at a temperature of less than or equal to 45° C., chosen from esters of at least one polyol chosen from the group formed by a polyethylene glycol comprising from 1 to 60 ethylene oxide units, sorbitan, glycerol comprising from 2 to 30 ethylene oxide units, and polyglycerols comprising from 2 to 12 glycerol units, and of at least one fatty acid comprising at least one saturated or unsaturated, linear or branched C₈-C₂₂ alkyl chain,
mixed esters of fatty acid or of fatty alcohol, of carboxylic acid and of glycerol,
fatty acid esters of sugars and fatty alkyl ethers of sugars,
surfactants that are solid at a temperature of less than or equal to 45° C., chosen from fatty acid esters of glycerol, fatty acid esters of sorbitan and oxyethylenated fatty acid sters of sorbitan, ethoxylated fatty acid ethers and ethoxylated fatty acid esters,
block copolymers of ethylene oxide (A) and of propylene oxide (B), and
silicone surfactants.

7. Cosmetic composition according to claim 1, wherein the nonionic surfactant (b) is chosen from:
polyethylene glycol isostearate or oleate (8 to 10 mol of ethylene oxide),
polyethylene glycol isocetyl, behenyl ether or isostearyl ether (8 to 10 mol of ethylene oxide),
polyglyceryl monolaurate or dilaurate comprising 3 to 6 glycerol units,
polyglyceryl mono(iso)stearate comprising 3 to 6 glycerol units,
polyglyceryl monooleate comprising 3 to 6 glycerol units, and
polyglyceryl dioleate comprising 3 to 6 glycerol units.

8. Cosmetic composition according to claim 1, in which the nonionic surfactant (b) is chosen from polyglyceryl fatty acid esters, preferably esters of a fatty acid and of polyglycerol comprising 70% or more of polyglycerol in which the degree of polymerization is 4 or more.

9. Cosmetic composition according to claim 1, in which the amount of nonionic surfactant (b) is in the range from 0.1% to 30% by weight relative to the total weight of the composition.

10. Cosmetic composition according to claim 1, in which the ratio of the nonionic surfactant (b) to the oil (a) is from 0.25 to 6, preferably from 0.3 to 3 and more preferably from 0.4 to 1.5.

11. Cosmetic composition according to claim 1, in which the C-glycoside compound (c) is represented by the following formula: 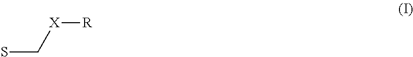
in which:
R represents a saturated or unsaturated C₁-C₁₀ and in particular C₁-C₄ alkyl radical, which may be optionally substituted with at least one radical chosen from OH, COOH, Y and COOR″₂ with R″₂ being a saturated C₁-C₄ alkyl radical, Y denotes a phenyl radical or a heterocycle, optionally substituted with 1 to 5 (ORₐ) groups,
S represents a monosaccharide or a polysaccharide comprising up to 20 sugar units, in particular up to 6 sugar units, in pyranose and/or furanose form and of the L and/or D series, it being possible for said monosaccharide or polysaccharide to be substituted with a hydroxyl group which must be free, and optionally with one or more optionally protected amine functions, and
X represents a radical chosen from the following groups: —CO—, —CH(OR′)—, —CH (NH₂)—, CHNR_{b}R_{c}; CHNHOR_{d}, —C(OR′)—, —C(NH₂)—, CNR_{b}R_{c}; CNHOR_{d} —CH(NHCH₂CH₂CH₂OH)—, —CH(NHPh)— and —CH(CH₃)—,
R′ denotes:
a hydrogen atom;
a saturated linear C1-C18 alkyl radical,
an unsaturated linear C2-C18 alkyl radical,
a saturated or unsaturated branched C3-C18 alkyl radical,
a saturated or unsaturated C5 or C6 cyclic radical,
a linear or branched, saturated or unsaturated C2-C18, or saturated or unsaturated C5 or C6 cyclic acyl radical.
Rₐ denotes:
a hydrogen atom
a linear or branched C1-C4 alkyl radical, or a linear or branched unsaturated C3-C4 hydrocarbon-based radical
a linear or branched C2-C18 acyl or linear or branched C2-C18 alkenylcarbonyl radical
R_{b} denotes:
a hydrogen atom
a linear C2-C18 or branched C3-C18 alkyl radical, or a linear or branched unsaturated C3-C4 hydrocarbon-based radical or a radical —CH(Z₁)—CO₂Z₂ in which
Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT, 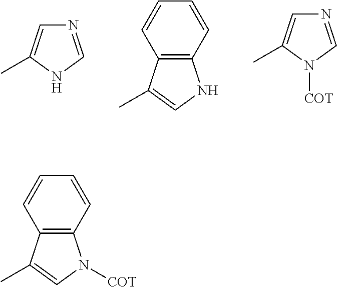
and/or interrupted with a group —NH—, —N—(COT)— or —S— with T denoting a linear or branched C1-C6 or cyclic C3-C6 alkyl radical.
and Z₂ denotes a hydrogen atom or a linear C1-C6 alkyl radical
R_{c} denotes:
a hydrogen atom
a linear C1-C4 or branched C3-C4 alkyl radical, or a linear or branched unsaturated C3-C4 hydrocarbon-based radical, the said radical being optionally substituted with a phenyl group.
Rd denotes:
a hydrogen atom
a linear C1-C18 or branched C3-C18 alkyl radical, or a linear or branched unsaturated C3-C18 hydrocarbon-based radical, the said radical being optionally substituted with a phenyl group,
the bond S—CH₂—X represents a bond of C-anomeric nature, which may be α or β,
and also the cosmetically acceptable salts thereof, solvates thereof, and optical and geometrical isomers thereof.

12. Cosmetic composition according to claim 1, in which the C-glycoside compound (c) is represented by the following formula: 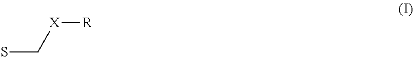
in which:
R denotes an unsubstituted linear C₁-C₄ alkyl radical, especially C₁-C₂, in particular methyl;
S represents a monosaccharide as described previously, chosen in particular from D-glucose, D-xylose, N-acetyl-D-glucosamine and
L-fucose, and in particular D-xylose;
X represents a group chosen from —CO—, —CH(OH)— and —CH(NH₂)— and preferentially a group —CH(OH)—.

13. Cosmetic composition according to claim 1, in which the C-glycoside compound (c) is C-β-D-xylopyranoside-2-hydroxypropane or C-α-D-xylopyranoside-2-hydroxypropane, and preferably C-β-D-xylopyranoside-2-hydroxypropane.

14. Cosmetic composition according to claim 1, in which the C-glycoside compound (c) is represented by formula (II) below: 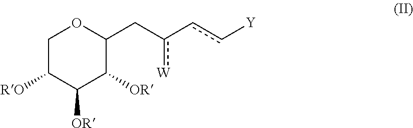
in which:
the compounds of formula (II) are xylose derivatives
Y denotes a phenyl radical or a heterocycle, optionally substituted with 1 to 5 groups (ORₐ)
W═—OR′; (═O); NR_{b}R_{c}; NHOR_{d}
R′ denotes:
a hydrogen atom;
a saturated linear C1-C18 alkyl radical,
an unsaturated linear C2-C18 alkyl radical,
a saturated or unsaturated branched C3-C18 alkyl radical,
a saturated or unsaturated C5 or C6 cyclic radical,
a linear or branched, saturated or unsaturated C2-C18, or saturated or unsaturated C5 or C6 cyclic acyl radical.
Rₐ denotes:
a hydrogen atom
a linear or branched C1-C4 alkyl radical, or a linear or branched unsaturated C3-C4 hydrocarbon-based radical
a linear or branched C2-C18 acyl or linear or branched C2-C18 alkenylcarbonyl radical
when Y denotes a phenyl radical or a heterocycle substituted with 2 to 5 groups (ORₐ), two adjacent groups ORₐ may together form a divalent radical —O—CH₂—O
with the proviso that when W═OH, the compound does not comprise an ethylenic double bond alpha to the carbon bearing this OH
R_{b} denotes:
a hydrogen atom
a linear C2-C18 or branched C3-C18 alkyl radical, or a linear or branched unsaturated C3-C4 hydrocarbon-based radical or a radical —CH(Z₁)—CO₂Z₂ in which
Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT, 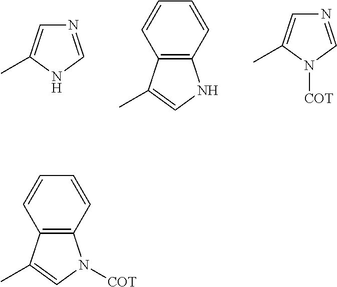
and/or interrupted with a group —NH—, —N—(COT)— or —S— with T denoting a linear or branched C1-C6 or cyclic C3-C6 alkyl radical.
and Z₂ denotes a hydrogen atom or a linear C1-C6 alkyl radical
R_{c} denotes:
a hydrogen atom
a linear C1-C4 or branched C3-C4 alkyl radical, or a linear or branched unsaturated C3-C4 hydrocarbon-based radical, the said radical being optionally substituted with a phenyl group.
Rd denotes:
a hydrogen atom
a linear C1-C18 or branched C3-C18 alkyl radical, or a linear or branched unsaturated C3-C18 hydrocarbon-based radical, the said radical being optionally substituted with a phenyl group,
and also the cosmetically acceptable salts thereof, solvates thereof such as hydrates, and stereoisomers thereof.

15. Composition according to claim 1, wherein the C-glycoside compound (c) is of formula (II) in which:
the compounds of formula (II) are xylose derivatives
Y denotes a phenyl radical or a heterocycle, optionally substituted with 1 to 3 groups (ORₐ)
W═—OR'; (═O); NR_{b}R_{c}; NHOR_{d}
R′ denotes:
a hydrogen atom,
a linear or branched, saturated or unsaturated C1-C4 alkyl radical,
a linear or branched C1-C6 acyl radical,
Rₐ denotes:
a hydrogen atom
a linear or branched C1-C4 alkyl radical,
a linear or branched C1-C6 acyl radical,
when Y denotes a phenyl radical or a heterocycle substituted with 2 or 3 groups (ORₐ), two adjacent groups ORₐ may together form a divalent radical —O—CH₂—O
with the proviso that when W═OH, the compound does not comprise an ethylenic double bond alpha to the carbon bearing this OH
R_{b} denotes:
a hydrogen atom
a linear C2-C8 or branched C3-C8 alkyl radical, or a radical —CH(Z₁)—CO₂Z₂ in which
Z₁ denotes a hydrogen atom or a linear or branched C1-C6, or saturated or unsaturated cyclic C3-C6 alkyl radical, the said radical being optionally substituted with at least one group chosen from ═NH, —NH₂, —N(T)₂, ═O, —OH, —OT, —SH, —ST, —CO₂T, phenyl, phenyl substituted with —OH or —OT, 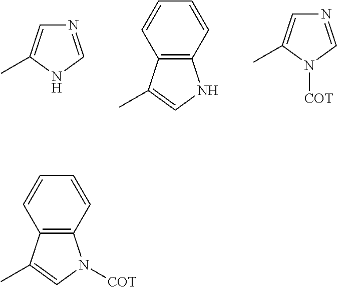
and/or interrupted with a group —NH—, —N—(COT)— or —S— with T denoting a linear C1-C6 or cyclic C5-C6 alkyl radical,
and Z₂ denotes a hydrogen atom or a linear C1-C6 alkyl radical
R_{c} denotes:
a hydrogen atom
a linear C1-C4 or branched C3-C4 alkyl radical, optionally substituted with a phenyl group,
Rd denotes:
a hydrogen atom
a linear C1-C4 or branched C3-C4 alkyl radical, the said radical being optionally substituted with a phenyl group,
and also the cosmetically acceptable salts thereof, solvates thereof, and stereoisomers thereof.

16. Composition according to claim 1, wherein the C-glycoside compound (c) is of formula (II) and is chosen from:
Compound 1.: (3R,4S,5R)-2-[2-hydroxy-4-(4-hydroxy-3-methoxyphenyl)butyl]tetrahydro-2H-pyran-3,4,5-triol 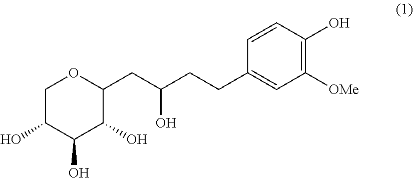 [SMILES: COc1cc(CCC(O)CC2OC[C@@H](O)[C@H](O)[C@H]2O)ccc1O]
Compound 2. 4-(4-hydroxy-3-methoxyphenyl)-1-[(3R,4S,5R)-3,4,5-trihydroxytetrahydro-2H-pyran-2-yl]butan-2-one 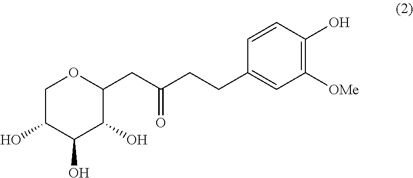 [SMILES: COc1cc(CCC(=O)CC2OC[C@@H](O)[C@H](O)[C@H]2O)ccc1O]
Compound 3. (3R,4S,5R)-2-[2-hydroxy-4-(4-hydroxyphenyl)butyl]tetrahydro-2H-pyran-3,4,5-triol 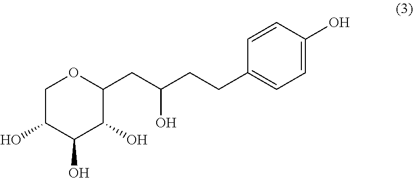 [SMILES: Oc1ccc(CCC(O)CC2OC[C@@H](O)[C@H](O)[C@H]2O)cc1]
Compound 4 4-(4-hydroxyphenyl)-1-[(3R,4S,5R)-3,4,5-trihydroxytetrahydro-2H-pyran-2-yl]butan-2-one 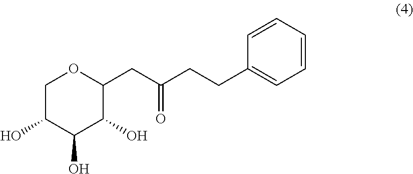 [SMILES: O=C(CCc1ccccc1)CC1OC[C@@H](O)[C@H](O)[C@H]1O]
Compound 5. (3R,4S,5R)-2-[2-(benzylamino)-4-(4-hydroxy-3-methoxyphenyl)butyl]tetrahydro-2H-pyran-3,4,5-triol 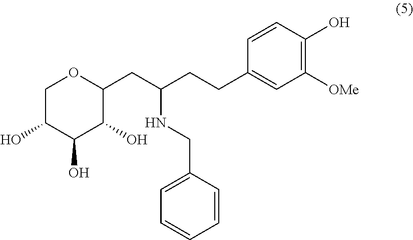 [SMILES: COc1cc(CCC(CC2OC[C@@H](O)[C@H](O)[C@H]2O)NCc2ccccc2)ccc1O]
Compound 6.
ethyl {[3-(4-hydroxy-3-methoxyphenyl)-1-{[(3R,4S,5R)-3,4,5-trihydroxytetrahydro-2H-pyran-2-yl]methyl}propyl]amino}(phenyl)acetate 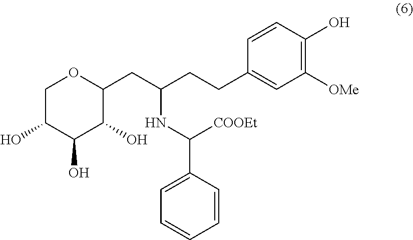 [SMILES: CCOC(=O)C(NC(CCc1ccc(O)c(OC)c1)CC1OC[C@@H](O)[C@H](O)[C@H]1O)c1ccccc1]
Compound 7. (3E)-4-phenyl-1-[(3R,4 S,5R)-3,4,5-trihydroxytetrahydro-2H-pyran-2-yl]but-3 -en-2-one 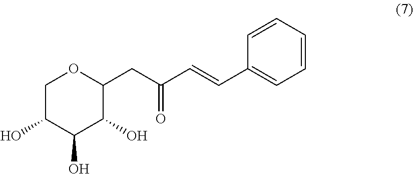 [SMILES: O=C(/C=C/c1ccccc1)CC1OC[C@@H](O)[C@H](O)[C@H]1O]
Compound 8. (3E)-4-(4-hydroxy-3,5-dimethoxyphenyl)-1-[(3R,4S,5R)-3,4,5-trihydroxytetrahydro-2H-pyran-2-yl]but-3 -en-2-one 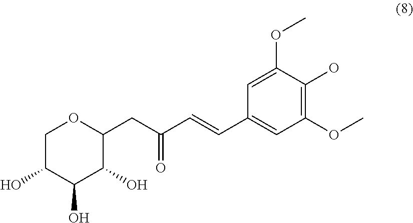 [SMILES: COc1cc(/C=C/C(=O)CC2OC[C@@H](O)[C@H](O)[C@H]2O)cc(OC)c1O]
Compound 9. (3R,4S,5R)-2-[2-hydroxy-4-(2-hydroxyphenyl)butyl]tetrahydro-2H-pyran-3,4,5-triol 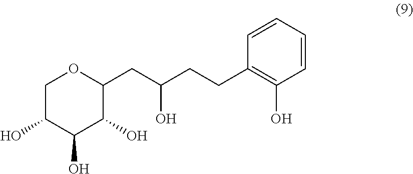 [SMILES: Oc1ccccc1CCC(O)CC1OC[C@@H](O)[C@H](O)[C@H]1O]
Compound 10: (3R,4S,5R)-2-[2-hydroxy-4-(3-hydroxy-4-methoxyphenyl)butyl]tetrahydro-2H-pyran-3,4,5-triol 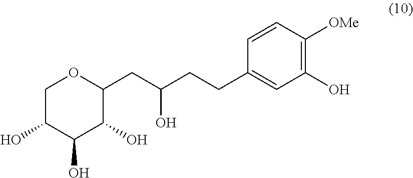 [SMILES: COc1ccc(CCC(O)CC2OC[C@@H](O)[C@H](O)[C@H]2O)cc1O]
Compound 11. (3R,4S,5R)-2-[2-hydroxy-4-(2,4-di-hydroxyphenyl)butyl]tetrahydro-2H-pyran-3,4,5-triol 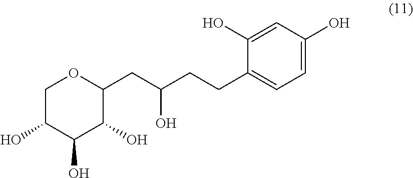 [SMILES: Oc1ccc(CCC(O)CC2OC[C@@H](O)[C@H](O)[C@H]2O)c(O)c1]
Compound 12. (3R,4S,5R)-2-[2-hydroxy-4-(3-ethoxy-4-hydroxyphenyl)butyl]tetrahydro-2H-pyran-3,4,5-triol 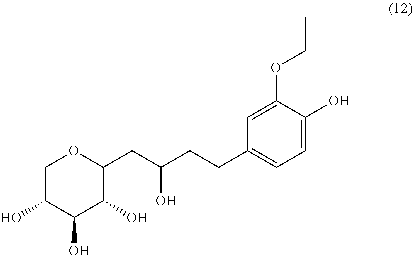 [SMILES: CCOc1cc(CCC(O)CC2OC[C@@H](O)[C@H](O)[C@H]2O)ccc1O]
Compound 13. 5,9-anhydro-1,2,4-trideoxy-1-pyridin-3 -yl-D-xylononitol 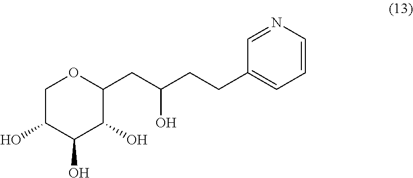 [SMILES: OC(CCc1cccnc1)CC1OC[C@@H](O)[C@H](O)[C@H]1O]
Compound 14. (3R,4S,5R)-2-[(2E)-2-(methoxyimino)-4-phenylbutyl]tetrahydro-2H-pyran-3,4,5-triol 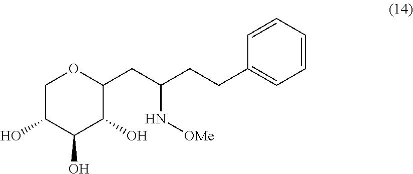 [SMILES: CONC(CCc1ccccc1)CC1OC[C@@H](O)[C@H](O)[C@H]1O]
Compound 15. 5,9-anhydro-1,2,4-trideoxy-7-O-pentanoyl-1-phenyl-D-xylonon-3-ulose 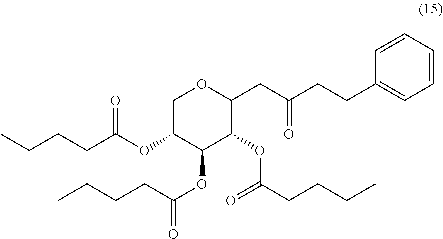 [SMILES: CCCCC(=O)O[C@H]1[C@H](OC(=O)CCCC)COC(CC(=O)CCc2ccccc2)[C@@H]1OC(=O)CCCC]
Compound 16. 5,9-anhydro-1,2,4-trideoxy-1-(3,4,5-trimethoxyphenyl)-D-xylononitol

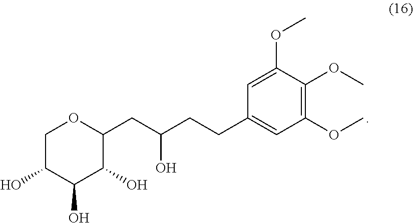 [SMILES: COc1cc(CCC(O)CC2OC[C@@H](O)[C@H](O)[C@H]2O)cc(OC)c1OC]

17. Composition according to claim 1, wherein the C-glycoside compound (c) is of formula (II) and is chosen from compounds (1) and (11).

18. Cosmetic composition according to claim 1, in which the amount of C-glycoside compound (c) is in the range from 0.01% to 20% by weight relative to the total weight of the composition.

19. Cosmetic composition according to claim 1, also comprising at least one nonionic surfactant other than (b) above and/or at least one ionic surfactant.

20. Cosmetic composition according to claim 1, also comprising at least one polyol.

21. Cosmetic composition according to claim 1, in which the cosmetic composition is in the form of an O/W emulsion, and the oil (a) is in the form of droplets with a numerical mean particle size of 300 nm or less.

22. Non-therapeutic process for treating the skin, the hair, mucous membranes, the nails, the eyelashes, the eyelids and/or the scalp, wherein the cosmetic composition according to claim 1 is applied to the skin, the hair, mucous membranes, the nails, the eyelashes, the eyelids or the scalp.

23. Use of the cosmetic composition according to claim 1 as or in care products and/or washing products and/or makeup products and/or makeup-removing products for bodily and/or facial skin and/or mucous membranes and/or the scalp and/or the hair and/or the nails and/or the eyelashes and/or the eyelids.
